SEQUENCE LISTING (2 sequences)

SEQ ID NO: 1 - protein, 20 aa, Artificial Sequence
  SILY is a synthesized amino acid sequence
RRANAALKAGELYKSILYGC

SEQ ID NO: 2 - protein, 21 aa, Artificial Sequence
  ZSILY is a synthesized amino acid sequence
ZRRANAALKAGELYKSILYGC

(19) United States
(12) Patent Application Publication
(10) Pub. No.: US 2015/0044259 A1
(43) Pub. Date: Feb. 12, 2015

(54) SCAFFOLD FOR ENHANCED NEURAL TISSUE REGENERATION

(71) Applicant: Mauris N. Desilva, Austin, TX (US)

(72) Inventor: Mauris N. Desilva, Austin, TX (US)

(21) Appl. No.: 13/962,667

(22) Filed: Aug. 8, 2013

(51) Int. Cl. A61K 38/18; A61K 9/00

(52) U.S. Cl. CPC A61K 38/185; A61K 9/00
USPC 424/400; 514/8.4

(57) ABSTRACT

This application discloses a scaffold for promoting the growth of a nerve in a mammal while minimizing clumping, which comprises a support structure having an elongate opening formed therein and configured for placement around a damaged region of a nerve and a physiologically acceptable matrix composition in said opening, said matrix composition comprising a Poly-D Lysine (PDL) and a peptidoglycan, and Nerve Growth Factor (NGF).

### CROSS REFERENCE TO RELATED APPLICATIONS

This application claims benefit of U.S. Provisional Application No. 61/681,581, filed Aug. 9, 2013. That application is incorporated herein by reference in its entirety.

### STATEMENT REGARDING FEDERALLY SPONSORED RESEARCH OR DEVELOPMENT

This invention was made with Government support under funding Work Unit Number G1026 by Naval Medical Research Unit San Antonio. The Government has certain rights in the invention.

### TECHNICAL FIELD

This invention is generally in the field of tissue engineering, and more particularly pertains to synthetic scaffold materials, and methods useful in directing tissue growth in vivo or ex vivo while reducing scar tissue formation and/or cell clumping.

### BACKGROUND

Nervous tissue damage is one of the most serious injuries suffered by US troops during combat. Medical procedures available today have limited success in peripheral nerve reconstruction, and there are no procedures available for repairing the central nervous system (CNS). The main challenge faced by the medical professionals is the formation of improper glial scar, which prevents neural regeneration.

Glial scar formation or gliosis is a reactive cellular process involving astrogliosis that occurs after injury to the central nervous system. The formation of glial scar is the body's natural mechanism to protect, which begin the healing process of the nervous system. In the context of neurodegeneration, formation of the glial scar has been shown to have both beneficial and detrimental effects. Absence of the glial scar has been linked to the impairments in the repair of blood brain barrier. Glial scar formation is associated with rapid proliferation and aggregation of astrocytes. Astrocytes, the most abundant star-shaped glial cells in the brain and spinal cord, secrete many neuro-developmental inhibitor molecules, which can prevent complete physical and functional recovery of the central nervous system after injury. For example, the heavy proliferation of astrocytes modifies the extracellular matrix surrounding the damaged brain region by secreting molecules such as laminin, fibronectin, tenascin C, and proteoglycans, which are important modulators of neuronal outgrowth. Thus, Glial scar formation is the main challenge to overcome as researchers search for a way to heal CNS injuries.

Collagen gels, form naturally under correct physiological conditions, have been widely used as scaffolds in tissue engineering. Collagen molecules can provide a three-dimensional environment by forming a periodic D-banding structure of networked collagen fibrils, which contains biological and mechanical conditions required for cellular activity. Attempts have been made to alter cellular responses by incorporating other extracellular matrix molecules, such as glycolsaminoglycans (GAGs) and proteoglycans (PGs), which can alter both fibrillogenesis and collagen organization. For example, Decorin is a small leucine-rich PG composed of a protein core and a dermatan sulfate (DS) side chain. The decorin core binds to collagen with high affinity in the nanomolar range. In vitro, the decorin-collagen interactions are shown to delay fibrillogenesis and enhance mechanical integrity of collagen structures [5].

Cells, scaffold and growth factor are key components to regenerate new tissues. Purdue University has developed novel peptidoglycans [5-7]. These peptidoglycans have been shown to bind to collagen, and regulate collagen fibrillogenesis, reduce dermal scarring in vivo, inhibit platelet binding to collagen in vivo, and suppress neointimal hyplerplasia following balloon angioplasty in vivo [5, 6]. These peptidoglycans also bind growth factors, which are important in wound healing, and enhance endothelial cell proliferation and migration, while inhibit MMP mediated collagen degradation[7]. They rapidly bind and persist in collagen tissue scaffold without chemical modification, and enhance keratinocyte proliferation. Peptidoglycan has been shown to play an important role in skin regeneration and scar reduction.

Fibrin is a fibrous, non-globular protein involved in the clotting of blood. Fibrin clots are formed as a reaction to peripheral nervous system injury. However, with CNS injury fibrin clots do not form. Fibrin scaffold is a network of protein that holds together, and supports a variety of living tissues. It is produced naturally by the body after injury, but can also be engineered in the lab. Fibrin scaffolds are shown to be helpful in repairing injuries to the urinary tract, liver, lung, spleen, kidney, and heart. Fibrin scaffolds have also been used to fill bone cavities, repair neurons, heart valves, vascular grafts and the surface of the eye. Fibrin scaffold is an important element in tissue engineering. In particular, fibrin is an attractive matrix for neural tissue regeneration because it seems to be correlated with PNS nerve regeneration[1]. It is advantageous when compared to synthetic polymers and collagen gels taking into consideration of cost, inflammation, immune response, toxicity and cell adhesion. Fibrin gels can be utilized as tissue scaffolds to provide cells with an attractive environment for growth and improved viability.

Similar to Fibrin gel, the Rat phechromocytoma cell line, also known as the PC12 cell line, is also used in nervous tissue regeneration research. PC 12 is an excellent model for neurons because they differentiate into neural cells in the presence of Nerve Growth Factor (NGF). However, they have the tendency to cluster in large groups when cultured, which leads to cell death. Previous research has involved culturing PC12 cells in 3D gels to study neurite outgrowth [1]. Current technology lacks methods for inhibiting PC12 cell clustering. The objectives of this work are to develop novel technology for enhance nervous tissue engineering and scar reduction.

### DESCRIPTIONS OF FIGURES

FIG. 1: PC12 Cell clump sizes (Example 1).

FIG. 2: PC12 Cell viability (Example 1).

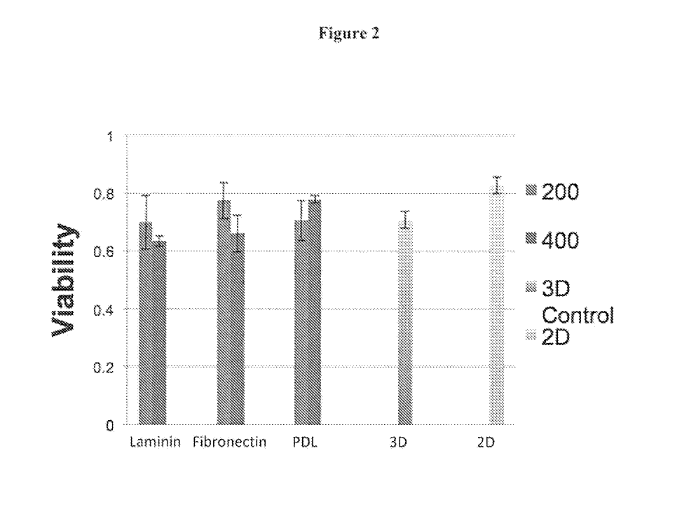

FIG. 3: PC12 Cell proliferation (Example 1).

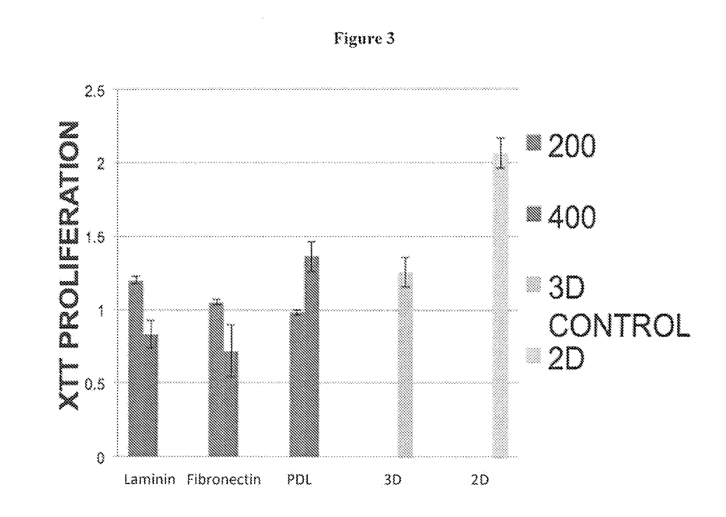

FIG. 4: Experimental Design for Cell migration Study using ADSC

FIG. 5: Cell Viability ADSC

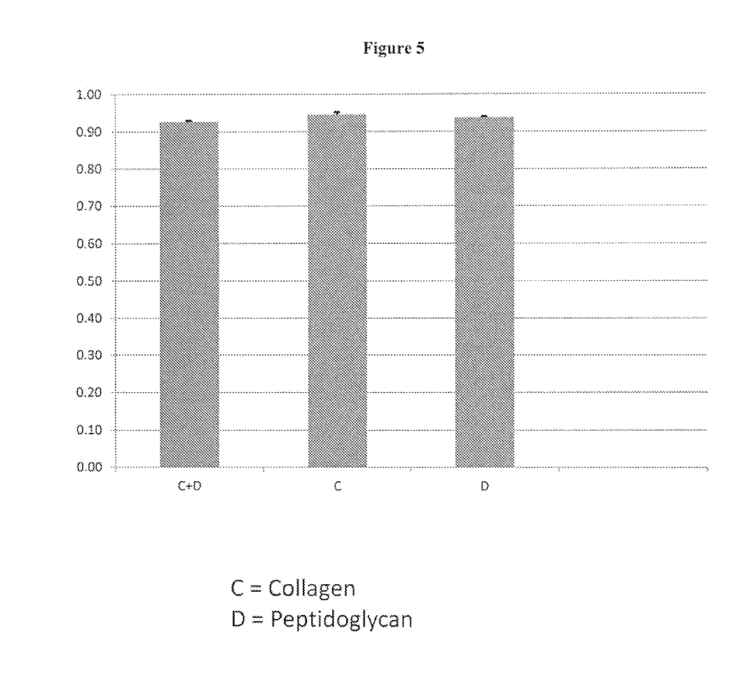

FIG. 6: Cell proliferation ADSC

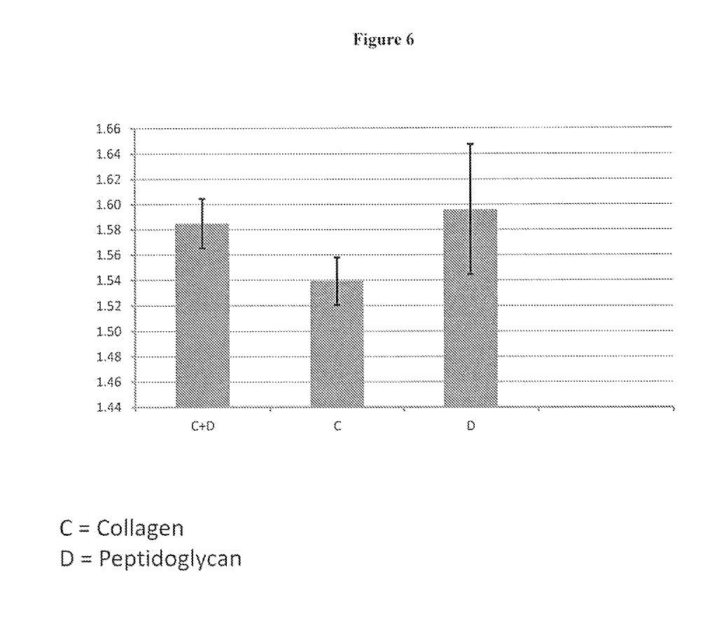

### DESCRIPTIONS OF THE INVENTION

The disclosures of all reference cited herein are hereby incorporated by reference herein in their entirety.

One of the main purposes of using a biomaterial in tissue regeneration is to provide a surrogate extracellular matrix (ECM) for cells to attach and grow. Specific interactions to ECM binding sites through cell receptors are important in maintaining proper cell function (Ingber D., Curr Opin Cell Biol 1991; 3(5):841-8; Tooney P. A. et al., Immunol Cell Biol 1993; 71(2):131-9; Jockusch B. M. et al., Annu Rev Cell Dev Biol 1995; 11:379-416; Ruoslahti E., Annu Rev Cell Dev Biol 1996; 12:697-715). Cells attach to the ECM through more than 20 known integrin receptors, more than half of which bind to the Arginine-Glycine-Aspartic Acid (RGD) peptide motif (Ruoslahti E., Annu Rev Cell Dev Biol 1996; 12:697-715).

Through the use of biomaterials that are both neuroconductive and neuroinductive, regeneration across large nerve defects may be possible. Subjects to be treated by the present invention include both human and animal subjects, particularly mammalian subjects such as dogs, cats, horses, cattle, mice, monkeys, baboons, etc., for both human and veterinary medicine purposes and drug and device development purposes.

Nerves to be treated by the methods of the invention include central nerve afferent and peripheral nerves such as somatic nerves, sensory-somatic nerves (including the cranial and spinal nerves), and autonomic nerves, which include sympathetic nerves, and parasympathetic nerves. Examples of nerves to be treated include, but are not limited to, cranial nerves, spinal nerves, nerves of the brachial plexus, nerves of the lumbar plexus, musculocutaneous nerve, femoral nerve, obturator nerve, sciatic nerve, the intercostal nerves, subcostal nerve, ulnar nerve, radial nerve, median nerve, pudendal nerve, saphenous nerve, common peroneal nerve, deep peroneal nerve, superficial peroneal nerve, and tibial nerve.

Damaged regions of nerves to be treated by the invention include those that have been subjected to a traumatic injury, such as crushed regions and severed (including fully and partially severed) regions, as well as nerves damaged in the course of a surgical procedure, e.g., as necessary to achieve another surgical goal and due to certain diseases such as Diabetes and Cancer. Damaged regions also include nerve regions that have degenerated due to a degenerative nerve disorder or the like, creating a “bottleneck” for axonal activity that can be identified by techniques such as electromyography and treated by use of the methods and devices of the present invention.n an embodiment of the present invention. A scaffold is used to promote the growth of a nerve in a mammal while reducing cell clumping clumping and reducing scar formation.

The physician first encases the damaged region of the nerve in a scaffold. The scaffold for promoting the growth of a nerve in a mammal, comprises a support structure having an elongate opening formed therein and configured for placement around a damaged region of a nerve; and a physiologically acceptable matrix composition in said opening, said matrix composition comprising a Poly-D Lysine (PDL) and a peptidoglycan each in an amount effective amount to promote nerve growth while reducing cell clumping and scar formation. An example of the peptidoglycan is a decorin, which is a small leucine-rich PG composed of a protein core and a dermatan sulfate side chain. Dermatan sulfate side chain of the decorin may be covalently bonded to an amino acid sequence set forth in SEQ ID:1 (SILY) or a an amino acid sequence set forth in SEQ ID:2 (DSILY). The preparation of SILY, and DS-SILY and their binding to collagen is taught in articles by Paderi et al[5-7], which are hereby incorporated by reference. Briefly, Peptide RRANAALKAGELYKSILYGC (SILY) was purchased from Genscript (Piscataway, N.J.). Briefly, peptidoglycan DS-SILY may be synthesized by coupled oxDS to the heterobifunctional crosslinker PDPH forming DS-PDPH. Excess PDPH was removed by size-exclusion chromatography and DS-PDPH was reacted with peptide SILY yielding the collagen-binding synthetic peptidoglycan DS-SILY. The peptidoglycan was separated from excess free peptide by size exclusion chromatography using MilliQ running buffer. DS-SILY was lyophilized and stored at −20° C. until further testing.

The matrix composition further comprises one or more of biomolecule, including but not limited to Polylysine, Laminin and a nerve growth factor (NGF). As used herein, “NGF” includes molecules that promote the regeneration, growth and survival of nervous tissue. Examples of growth factors include, but are not limited to, nerve growth factor (NGF) and other neurotrophins, platelet-derived growth factor (PDGF), erythropoietin (EPO), thrombopoietin (TPO), myostatin (GDF-8), growth differentiation factor-9 (GDF9), basic fibroblast growth factor (bFGF or FGF2), epidermal growth factor (EGF), hepatocyte growth factor (HGF), granulocyte-colony stimulating factor (G-CSF), and granulocyte-macrophage colony stimulating factor (GM-CSF). There are many structurally and evolutionarily related proteins that make up large families of growth factors, and there are numerous growth factor families, e.g., the neurotrophins (NGF, BDNF, and NT3). The neurotrophins are a family of molecules that promote the growth and survival of nervous tissue. Examples of neurotrophins include, but are not limited to, nerve growth factor (NGF), brain-derived neurotrophic factor (BDNF), neurotrophin 3 (NT-3), and neurotrophin 4 (NT-4). See U.S. Pat. No. 5,843,914 to Johnson, Jr. et al.; U.S. Pat. No. 5,488,099 to Persson et al.; U.S. Pat. No. 5,438,121 to Barde et al.; U.S. Pat. No. 5,235,043 to Collins et al.; and U.S. Pat. No. 6,005,081 to Burton et al.

For example, nerve growth factor (NGF) can be added to the keratin matrix composition in an amount effective to promote the regeneration, growth and survival of nervous tissue. The NGF is provided in concentrations ranging from 0.1 ng/ml to 1000 ng/ml. More preferably, NGF is provided in concentrations ranging from 1 ng/ml to 100 ng/ml, and most preferably 10 ng/ml to 100 ng/ml. See U.S. Pat. No. 6,063,757 to Urso.

As used herein, “support structure,” “scaffold,” etc., is any suitable structure into which a damaged nerve may be placed, and can support or contain matrix material during nerve regeneration. In general, the structure is formed of a physiologically acceptable material. In some embodiments the support structure has an elongate opening formed therein, such as a conduit structure in the shape of a tube having a single longitudinal opening, or any suitable shape, including square, hexagonal, triangular, etc., with any number of openings (such as fibrils as described below) may be used. Other examples of embodiments suitable to carry out the present invention will be apparent to those skilled in the art. For example, the support structure can be in the shape of a gutter, with or without an additional top piece. The gutter support structure may also have a top piece, placed in such a way as to “sandwich” the damaged nerve between the two pieces.

The material from which the support structure is formed can be bioabsorbable or inert (that is, non-bioabsorbable). Any bioabsorbable material may be used, including but not limited to natural materials such as collagen, laminin, firbin gel, alginate and combinations thereof, etc., as well as synthetic materials such as poly(lactide), poly(glycolide), poly(caproic acid), combinations thereof, etc. Materials may be polymeric or non-polymeric. Examples of suitable support structures include, but are not limited to, the artificial neural tubes described in U.S. Pat. Nos. 6,589,257 and 6,090,117 to Shimizu, the guide tubes described in U.S. Pat. No. 5,656,605 to Hansson et al., the tubular prostheses described in U.S. Pat. No. 4,662,884 to Stensaas, the elastomeric devices described in U.S. Pat. No. 5,468,253 to Bezwada et al., and the biopolymer rods with oriented fibrils (which fibrils then form a plurality of elongate openings or tubes containing the matrix described herein) as described in U.S. Pat. No. 6,461,629 to Tranquillo et al.

Other options for configuration of the support structure include having a longitudinal slit to facilitate the positioning of the structure around a damaged nerve, such as described in U.S. Pat. No. 4,662,884 to Stensaas. The interior wall portion of the support structure may optionally be patterned to facilitate or guide regeneration, as described in U.S. Pat. No. 6,676,675 to Mallapragada et al. The elongate opening may optionally contain guiding filaments dispersed within the matrix and extending along the longitudinal dimension of the support structure, as described in U.S. Pat. No. 5,656,605 to Hansson et al. The support structure may optionally include one, two or more electrodes connected to or otherwise operatively associated therewith to aid in applying an electric field to the nerve to facilitate regeneration.

The support structure may be packaged in sterile form in a sterile aseptic container. The sterile matrix composition may be provided in the support structure as packaged, in hydrated or dehydrated form (for subsequent hydration with a suitable solution such as sterile physiologically acceptable saline solution once opened for use), or the matrix packaged separately (in hydrated or dehydrated form, in a vial, syringe, or any other suitable container) for administration into the support structure before or during the time of use.

In some embodiments, the support structure is positioned around the damaged region of the nerve, and matrix is added as necessary. This may be carried out by any suitable technique, such as by opening the structure (e.g., along a longitudinal slit) and then enclosing it around the damaged portion of the nerve, by inserting each stump (proximal, distal) of a severed nerve into opposite ends of the support structure opening, etc. Sutures, surgical adhesives, staples, clasps, prongs formed on the inner surface of the support structure at each end thereof, or any other suitable technique may be used to secure the nerve in place.

Surgical procedures can otherwise be carried out in accordance with known techniques, including but not limited to those described in U.S. Pat. Nos. 6,589,257 and 6,090,117 to Shimizu, U.S. Pat. No. 5,656,605 to Hansson et al., U.S. Pat. No. 4,662,884 to Stensaas, U.S. Pat. No. 5,468,253 to Bezwada et al., and U.S. Pat. No. 6,676,675 to Mallapragada et al.

### Example 1

### Modified Fibrin Scaffold Reduces Cell Clumping

Cells were suspended in fibrin gel by combining 50 μl 12.5 mg/ml fibrinogen and 50 μl 62.5 mg/ml thrombin. To reduce clumping, 200 ng/ml and 400 ng/ml of laminin, fibronectin and Poly-D Lysine (PDL) were incorporated in the fibrin gel. A monolayer was prepared on a PDL coated substrate as a control.

### Cell Culture

The PC12 cells were cultured at 37° C. and 5% CO2 in Dulbecco's Modified Eagle's medium (Invitrogen) supplemented with Ham's F12, Horse Serum (ATCC), Bovine calf serum (ATLANTA BIOLOGICALS®, Norcross, Ga.), antibiotic (penicillin/streptomycin INVITROGEN™, Carlsbad, Calif.), and Glutamine (SIGMA-ALDRICH®, St. louis Missouri). Cells were passaged every 3-4 days. PC 12 cells were allowed to migrate for 3 days, during which the media was not changed.

### Fibrin Formation

A 1.65 ml master solution of fibrinogen was first created by diluting Human Fibrinogen (SIGMA-ALDRICH®, St. louis Missouri) to 12.5 mg/ml using 1×TBS. The solution was then syringe filtered. A 1.55 ml master solution was created for thrombin by diluting Human Thrombin (SIGMA-ALDRICH®, St. louis Missouri) to 62.5 mg/ml with CaCl(AcrosOrganic). Fibrinogen was separtated into tubes each with 150 ul. In all but one tube, 200 ng/ml or 400 ng/ml of either laminin (SIGMA-ALDRICH®, St. louis Missouri), fibronectin, or poly-d lysine (PDL SIGMA-ALDRICH®, St. louis Missouri). 300 ul of Cells at a concentation of fifty thousand cells were added to thrombin. Fibrin gels were created by mixing 50 ul of the fibrinogen solution with 50 ul of the thrombin solution. The gels were allowed to polymerize for 10 minutes. 100 ul of media was used to cover each gel after polymeration. 3D cultures were left to incubate for three days in an environment with 5% CO2 at 37° C.

PC 12 cell cluster size was determined by counting the number of clusters and the average size of clusters in three random fields per condition at 20× magnification using phase microscopy. Proliferation was assayed with an XTT test. Viability was assayed using Fluorescein Diacetate (FDA) and Propidium Iodide (PI) to stain live and dead cells respectively. The fluorescent intensity was measured and used to calculate viability: FDA/PI+FDA. One-way ANOVA analysis was performed with post hoc adjustment for analytic comparison.

### Results

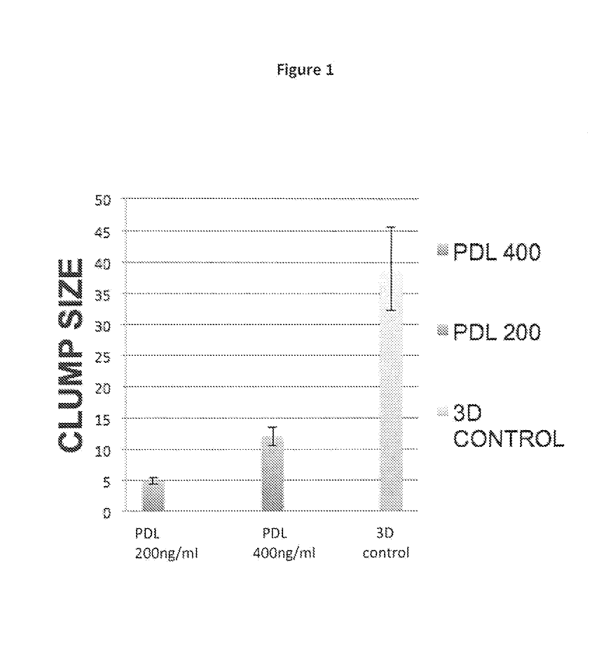

The incorporation of Laminin and Fibronectin had little impact on PC12 cell aggregation in comparison to the 3D control. As the amount of PDL was increased, the amount and size of clumps decreased. PC12 cells were clustered the least when the fibrin contained 400 ng/ml PDL. As illustrated in FIG. 1, the influence of PDL on PC12 cell clump size was statistically significant. However, the influence of 400 ng/ml versus 200 ng/ml of PDL was not statistically significant. The viability and proliferation of all the 3D cultures with adhesive molecules were not statistically different from the 3D control. However, the viability of the 400 ng/ml laminin condition was statistically different when compared to the 2D culture, demonstrating the need for improvement in fibrin gels as a PC12 cell environment. Proliferation demonstrated a statistically significant difference when comparing the 2D control and the 3D control. The proliferation of the 200 ng/ml fibronectin, 400 ng/ml fibronectin, 400 ng/ml laminin, and 200 ng/ml PDL conditions were statistically different from the 2D control. PDL was the only adhesive molecule that improved viability, and proliferation as the concentration increased while also decreasing clump size.

In summary, PDL influences PC12 cell migration within fibrin gels, and assisted in spreading the cells throughout the gel rather than clumping. In the 3D cultures, viability and proliferation were not statistically affected by the adhesive molecules. However, future projects will work to improve both viability, and proliferation to maximized scaffold potential. Higher concentrations of PDL should be studied to see if they improve viability and proliferation, and lessen clump size. Fibrin gels with PDL will also be studied with NGF to see the effects of PDL on neurite outgrowth. Fibrin gel fibers will be modified with the addition of glial scar inhibiting molecules to minimize scar formation.

### Example 2

### Using Adipose Derived Stem Cells from Discarded Tissue/Liposuction and Neurons for Enhanced Regeneration

Cells, scaffold and growth factor are key components to regenerate new tissues. Purdue University has developed novel peptidoglycans [5-7]. These peptidoglycans have been shown to bind to collagen, and regulate collagen fibrillogenesis, reduce dermal scarring in vivo, inhibit platelet binding to collagen in vivo, and suppress neointimal hyplerplasia following balloon angioplasty in vivo [5, 6]. These peptidoglycans also bind growth factors, which are important in wound healing, and enhance endothelial cell proliferation and migration, while inhibit MMP mediated collagen degradation[7]. They rapidly bind and persist in collagen tissue scaffold without chemical modification, and enhance keratinocyte proliferation. Peptidoglycan has been shown to play an important role in skin regeneration and scar reduction.

Collagen coating is prepared by diluting 5 mg of collagen (source) with 25 μl of acetic acid (source) in 9.975 μl distilled water. Peptidoglycan coating is prepared by dissolving with 2.5 mg of Peptidoglycan in 1 ml PBS. 96 well plates were used for viability and proliferation studies. The plates are coated with collagen solution, only at 4 degrees Celsius overnight, and washed 3 times with HBSS. The plates are then divided into two groups. Half of the plates, a 100 microliter of 2.5 mg/ml D-Sily solution was added to the well. Incubate at 37 degrees for 10 min. Remove D-sily solution. For the third group, a 100 microliter of 2.5 mg/ml D-Sily solution was added to the half the well.

### Migration Studies

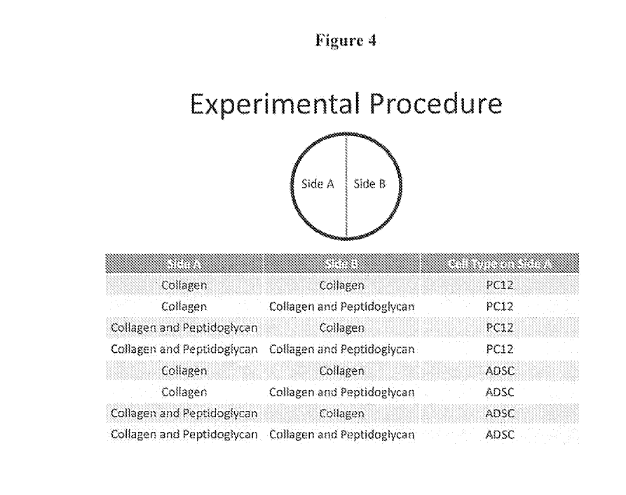

Collagen coating is prepared by diluting 5 mg of collagen (source) with 25 μl of acetic acid (source) in 9.975 μl distilled water. Peptidoglycan coating is prepared by dissolving with 2.5 mg of Peptidoglycan in 1 ml PBS. 35 mm glass-bottom dish were used for migration study. 50 k cells were added to one side of the plate, and incubate for 1 hr for cells to attach to the surface. PDMS divider is inserted. Media was added to one side to ensure that these is no leak. A 100 microliter of 2.5 mg/ml D-Sily solution was added to the half the well as shown in FIG. 4. Incubate at 37 degrees for 10 min. Remove D-sily solution. Remove divider add more media.

### Adipose Derived Stem Cells (ADSC) Culture

Warm growth medium in a water bath to 37° C. Aspirate medium and rinse culture with Hank's Balanced Salt Solution (HBSS). Aspirate HBSS and rinse cell monolayer with 750 μl 0.25% Trypsin-EDTA solution warmed to 37° C. Incubate flasks at room temperature, inspecting cells under the microscope periodically, and gently rocking the flasks to redistribute trypsin. ASCs usually detach within 2-3 minutes. If the cells are not rounded up, and coming off after 5 minutes, the flasks may be placed in the 37° C. incubator for 2-minute. Once the cells are rounded, gently tap the flasks to dislodge the cells. Add 5 ml of growth medium to stop trypsin action, and pipette gently to obtain a single cell suspension. Transfer cell suspension to a 15 ml centrifuge tube, and remove a 0.5 ml aliquot to count using an automated cell counter or a hemacytometer using trypan blue dye exclusion viability stain. Centrifuge cell suspension at 1900 rpm for 10 minutes. Aspirate medium and resuspend pellet in growth medium, pre-warmed in water bath at 37° C. Cells were cultured in 96 well plates with appropriate coating materials. The coating materials are assigned to plates according to the experimental design illustrated in FIG. 4.

PC12 and ADSC cells were placed on Side A, and through cell proliferation, migrates to side B. Cell morphology was monitored up to 21 days and evaluated using light microcopy.

### Results

Preliminary results show that Peptidoglycan did not negatively impact on cell viability andproliferation and in migration studies D-Sily enhanced cell migration. Also, the cells were able to keep longer in collagen gels with peptidoglycans.

Cell migration and proliferation from collagen to Collagen with modified Peptidoglycan (D-Sily) was enhanced compared to cell migration and proliferation from collagen to collagen. After two weeks leading edge of ADSC from collagen to collagen with modified Peptidoglycan (D-Sily) migrated all the way to the distal edge of the glass bottom dish (6 mm), when leading edge of cells have migrated only up to half the distance (3 mm). Collagen with addition of modified Peptidoglycan (D-Sily) encourages cells migration.

Cell migration and proliferation from collagen to Collagen with modified Peptidoglycan (D-Sily) was enhanced compared to cell migration and proliferation from collagen to collagen. After two weeks leading edge of ADSC from collagen to collagen with modified Peptidoglycan (D-Sily) migrated all the way to the distal edge of the glass bottom dish, when leading edges of cells have migrated only up to half the distance. Collagen with addition of modified Peptidoglycan (D-Sily) encourages cells migration.

### Prophetic Example 3

### Further Testing

In vitro and in vivo experiments and animal testing will be conducted to show that combination of biomolecules enhance tissue healing and drive the resident cells toward tissue regeneration rather than scar formation, and also, enhance the nerves to re-grow appropriately

### REFERENCES

1. Akassoglou et al., Journal of cell Biology Vol. 149, No. 5, p 1157-166, 2000.
2. Pittier, R. et al., Journal of Neurobiology, 63(1):1-14, 2005.
3. Diester et al., Journal of Biomaterial Science, Vol. 18, No. 8 pp. 983-997, 2007.
4. Chan, Odde, Journal of Cell Biology, Science, 322(5908):1687-91., 2008.
5. Paderi J E, Panitch A. Biomacromolecules.9(9):2562-6. Epub 2008 Aug. 5. Design of a synthetic collagen-binding peptidoglycan that modulates collagen fibrillogenesis.
6. Paderi J E, Stuart K, Sturek M, Park K, Panitch A. Biomaterials. 32(10):2516-23. j.biomaterials.2010.12.025. The inhibition of platelet adhesion and activation on collagen during balloon angioplasty by collagen-binding peptidoglycans. Epub 2011 Jan. 8.
7. Stuart K, Paderi J, Snyder P W, Freeman L, Panitch A. Collagen-binding peptidoglycans inhibit MMP mediated collagen degradation and reduce dermal scarring. PLoS One. 201; Epub 2011 Jul. 11.

1) A scaffold for promoting the growth of a nerve in a mammal, comprising:
a) a support structure having an elongate opening formed therein and configured for placement around a damaged region of a nerve; and
b) a physiologically acceptable matrix composition in said opening, said matrix composition comprising a Poly-D Lysine (PDL) and a peptidoglycan.

2) The scaffold of claim 1, wherein said peptidoglycan is a decorin.

3) The scaffold of claim 2, wherein a dermatan sulfate side chain of said decorin is covalently bonded to an amino acid sequence set forth in SEQ ID:1.

4) The scaffold of claim 2, wherein a dermatan sulfate side chain of said decorin is covalently bonded to an amino acid sequence set forth in SEQ ID:2.

5) The scaffold of claim 1, wherein said matrix composition further comprises one or more of biomolecules selected from the group consisting:
a) a Polylysine;
b) a Laminin; and
c) a nerve growth factor (NGF).

6) The scaffold of claim 1, wherein said support structure is formed from a bioabsorbable material.

7) The scaffold of claim 6, wherein said bioabsorable material selected from one or more of the group consisting of:
a) a fibrin gel; and
b) a collagen.

8) The scaffold of claim 1, wherein said support structure is formed from an inert polymeric material.

9) A method for promoting the growth of a nerve in a mammal, comprising: encasing a damaged region of said nerve in a support structure having an elongate opening therein; and administering a physiologically acceptable matrix composition into said opening, said matrix composition comprising said matrix composition comprising a Poly-D Lysine (PDL) and a peptidoglycan, each in an amount effective to promote the growth of said nerve at said damaged region while reducing cell clumping.

10) The method of claim 9, wherein said damaged region of said nerve is a crushed region.

11) The method of claim 9, wherein said damaged region of said nerve is a severed region having proximal and distal stumps, and said encasing step is carried out by placing the proximal and distal stumps of said nerve in said support structure.

12) The method of claim 11, wherein said nerve is a central nerve or a peripheral nerve selected from the group consisting of sensory-somatic nerves and autonomic nerve.

13) The method of claim 9, wherein said peptidoglycan is a decorin.

14) The scaffold of claim 13, wherein a dermatan sulfate side chain of said decorin is covalently bonded to an amino acid sequence set forth in SEQ ID:1.

15) The scaffold of claim 12, wherein a dermatan sulfate side chain of said decorin is covalently bonded to an amino acid sequence set forth in SEQ ID:2.

16) The scaffold of claim 9, wherein said matrix composition further comprises one or more of biomolecules selected from the group consisting
a) a Polylysine;
b) a Laminin; and
c) a Nerve Growth Factor (NGF).

17) The method of claim 9, wherein said support structure is formed from a bioabsorbable material.

18) The method of claim 10, wherein said bioabsorbable material selected from one or more of the group consisting of
a) a fibrin gel; or
b) a collagen.

19) The scaffold of claim 9, wherein said support structure is formed from an inert polymeric material.

20) A kit comprising:
a) a support structure having an elongate opening formed therein and configured for placement around a damaged region of a nerve;
b) a container, wherein, said support structure is packaged in said container sterile form; and
c) a physiologically acceptable matrix composition, wherein said matrix composition is sterile, and wherein said matrix composition comprises a Poly-D Lysine (PDL) and a peptidoglycan.

21) The kit of claim 20, wherein said peptidoglycan is a decorin.

22) The kit of claim 20, wherein a dermatan sulfate side chain of said decorin is covalently bonded to an amino acid sequence set forth in SEQ ID:1.

23) The kit of claim 20, wherein a dermatan sulfate side chain of said decorin is covalently bonded to an amino acid sequence set forth in SEQ ID:2.

24) The kit of claim 20, wherein said matrix composition further comprises one or more of biomolecules selected from the group consisting:
d) a Polylysine;
e) a Laminin; and
f) a nerve growth factor (NGF).

25) The kit of claim 20, wherein said support structure is formed from a bioabsorbable material.

26) The kit of claim 25, wherein said bioabsorable material selected from one or more of the group consisting of:
c) a fibrin gel; and
d) a collagen.

27) The kit of claim 25, wherein said support structure is formed from an inert polymeric material.
